(19) United States
(12) Patent Application Publication
(10) Pub. No.: US 2016/0356754 A1
(43) Pub. Date: Dec. 8, 2016

(54) FOOD FRESHNESS INDICATING LABEL

(71) Applicant: TOSHIBA TEC KABUSHIKI KAISHA, Tokyo (JP)

(72) Inventors: Ryozo AKIYAMA, Mishima Shizuoka (JP); Kenichi KOMIYA, Kawasaki Kanagawa (JP); Daisuke ISHIKAWA, Mishima Shizuoka (JP); Ryuichi ARAI, Numazu Shizuoka (JP); Naoko FUTAMI, Fuji Shizuoka (JP)

(21) Appl. No.: 15/239,944

(22) Filed: Aug. 18, 2016

Related U.S. Application Data
(62) Division of application No. 14/661,836, filed on Mar. 18, 2015.

(30) Foreign Application Priority Data
Mar. 20, 2014 (JP) 2014-058313

(51) Int. Cl. G01N 33/12; G01N 21/78

(52) U.S. Cl. CPC G01N 33/12; G01N 21/783

(57) ABSTRACT

A freshness indicating medium includes a base member having a first region and a second region. Both the first and second regions contain a reactive material that changes a state thereof by reaction with a food-derived material that is generated as the food product putrefies, such that, as an amount of the food-derived material increases, the reactive material in the first region changes the state before the reactive material in the second region does.

### CROSS-REFERENCE TO RELATED APPLICATION

This application is a division of U.S. patent application Ser. No. 14/661,836, filed on Mar. 18, 2015, which is based upon and claims the benefit of priority from Japanese Patent Application No. 2014-058313, filed Mar. 20, 2014, the entire contents of which are incorporated herein by reference.

### FIELD

Embodiments described herein relate to a food freshness indicating label with which freshness or a putrefaction state of a food product may be recognized.

### BACKGROUND

Whether or not a food product is safely consumable depends on how well the food product has been preserved. Thus, a use-by or consume-by date indicated on a package of the food product may not correctly indicate whether the food product is still safely consumable.

A freshness label may be employed to more reliably indicate whether or not the food product is safely consumable. The freshness label contains a food-derived bacteria and a solution that are mixed together, and a color of the solution changes as the food-derived bacteria propagates. A consumer can recognize the freshness of the food product by checking the color of the solution.

However, the color of the solution may not correctly indicate the actual freshness of the food product because there may be mismatch between the bacteria contained in the freshness label and the actual bacteria of the food product.

### DESCRIPTION OF THE DRAWINGS

FIG. 1 illustrates one example of a freshness indicating label according to an embodiment.

FIG. 2 illustrates a usage example of the freshness indicating label according to the embodiment.

FIG. 3A to FIG. 3C illustrates a freshness indicating label with which a region is recognizable in a non-visible spectrum according to another embodiment.

FIG. 4 is a cross-sectional view of a freshness indicating label according to another embodiment.

FIG. 5A and FIG. 5B are cross-sectional views of a freshness label according to another embodiment.

FIG. 6 is a cross-sectional view of a freshness indicating label according to another embodiment.

FIG. 7 is a plan view of a freshness indicating label according to another embodiment.

FIG. 8A and FIG. 8B illustrate a freshness indicating label according to another embodiment.

### DETAILED DESCRIPTION

One or more embodiments in this disclosure provide a freshness indicating label with which a user may easily know freshness or a putrefaction state of a food product without using a large-sized analyzer, may know the degree of freshness or a putrefaction state of the food product, and may know whether or not the food product is consumable or whether or not the food product may be processed depending on a state of freshness.

In general, according to one embodiment, a freshness indicating medium includes a base member having a first region and a second region. Both the first and second regions contain a reactive material that changes a state thereof by reaction with a food-derived material that is generated as the food product putrefies, such that, as an amount of the food-derived material increases, the reactive material in the first region changes the state before the reactive material in the second region does.

Hereinafter, this embodiment is explained in detail with reference to drawings.

### (Freshness Label)

A freshness label (freshness indicating medium) according to this embodiment basically includes following: a component B which reacts with a component A (hereinafter referred to as “putrefaction component A” or simply referred to as “component A”) generated due to a change in freshness or a putrefaction state of food; and a holding body C which holds the component B. The component B generates color or is decolored by reacting with the putrefaction component A. Alternatively, the reaction of the component B with the putrefaction component A may be detected using spectroscopic characteristics of light within a wavelength range outside a wavelength range of visible light.

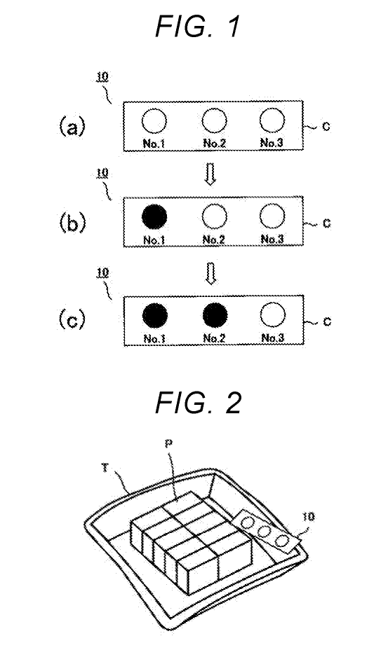

FIG. 1 illustrates one example of the freshness label according to this embodiment. A freshness label 10 is configured such that three circular regions No. 1, No. 2 and No. 3 which differ from each other in concentration of the contained component B are formed on a sheet which configures the holding body C. Out of these regions, the region No. 1 includes the highest content of component B (concentration of the contained component B), and the content of component B (concentration of the contained component B) is lower than the region No. 1 in the order of the region No. 2 and the region No. 3. Before the freshness label 10 is used, all regions No. 1 to No. 3 on the freshness label 10 have a color of yellow. When a putrefaction component A is generated, the color of the region No. 1 which includes the highest content of component B (concentration of the contained component B) tends to first change and hence, the detection of putrefaction at an initial level becomes possible. When the change in color of the region No. 3 is recognized, it can be determined that the putrefaction of a food is at a level where the food is not consumable. In this manner, the component B of the freshness label 10 is suitably selected component B in conformity with food, which is an object to which the freshness label 10 is applied; hence, a freshness level of the food may be understood in a stepwise manner.

(Putrefaction Component A)

As described above, the freshness label according to this embodiment indicates a quantity of putrefaction component A in a simplified manner, and allows a person to recognize freshness or a putrefaction level of food in a stepwise manner. Hereinafter, the putrefaction component A is explained.

In general, when a food is left without preservation, some change takes place in smell, external appearance, texture or taste along with a lapse of time, and, eventually, the food becomes unsuitable for eating. Such an adverse change in food is referred to as “degradation” or “deterioration.” In general, such a change is expressed as “food goes bad.” The degradation of food takes place due to insects, autolylsis, a chemical factor (oxidation or browning of fat) or a physical factor (damage such as scars or crushes) besides microorganisms. However, in many cases, food is deteriorated due to the propagation of microorganisms and becomes not consumable. Such deterioration is referred to as “putrefaction” in a broad definition of the term.

In some cases, “putrefaction” is distinguished from “deterioration” or “degradation.” That is, a process where protein in food is decomposed upon receiving an effect of microorganisms (putrefactive bacteria) so that a harmful material or odor is generated is referred to as “putrefaction.” On the other hand, a state where carbohydrate or grease in food is decomposed upon receiving an action from microorganisms so that food deteriorates flavor thereof and is not suitable for eating is referred to as “deterioration” or “degradation.” Main components of putrefactive odor may be various kinds of volatile amine such as ammonia or trimethylamine. Accordingly, it is useful to determine the quantity of volatile amine components to know the degree of putrefaction of food rich in protein like meat and fish.

In performing a quantitative analysis of a volatile amine component, volatile basic nitrogen (abbreviated as “VBN”) is measured. As criteria of a putrefaction state, a state where VBN is cumulated up to 30 mg % is set as initial putrefaction, and a state where VBN is 50 mg % or more is set as complete putrefaction. A microdiffusion method which uses a Conway unit as a method of measuring the VBN is known. However, this method has a drawback that a diffusion operation requires a time so that a quick inspection cannot be performed.

Further, while nitrogen compounds in food mainly take the form of protein, these nitrogen compounds are hydrolyzed into polypeptide, simple peptide, or an amino acid by enzymes of microorganisms or enzymes of food. Then, the amino acid is decomposed and metabolized due to a deamination reaction, transamination, a decarboxylation reaction or the like.

As amine which is generated from an amino acid, for example, histamine, tyramine, agmatine, putrescine, cadaverine, alanine, tryptamine, isobutylamine, isoamylamine and the like may be named. Hydrogen sulfide or mercaptan is generated from a sulphur-containing amino acid, indole, or skatole is generated from tryptophan, and cresol or phenol is generated from tyrosine.

(Component B which Reacts with Putrefaction Component A)

The component B which reacts with the putrefaction component A according to this embodiment may be suitably selected corresponding to the above-mentioned putrefaction component A.

For example, when a component having basicity such as amine is mainly detected as a putrefaction component A, a pigment whose color changes depending on pH (hereinafter also referred to as “pH color changing pigment”) may be used.

The freshness label according to this embodiment is basically configured such that a component B is held in the holding body C. By taking into account that the freshness label may be in contact with food to which the freshness label is applied or the like, it is desirable that the pH color changing pigment be a natural pigment having no or significantly low toxicity.

For the natural pH color changing pigment, for example, an anthocyanin pigment such as a red cabbage pigment, a purple sweet potato pigment, a grape skin pigment, a grape juice pigment, an elderberry pigment, a purple corn pigment, a perilla pigment or a hibiscus pigment may be used.

A quinone pigment, such as a cochineal pigment, a lac pigment, or a madder pigment, which becomes purple red color when the food is alkaline, or turmeric pigment which becomes a red-brown color when the food is alkaline may also be used. In addition to the above, a carotenoid pigment, a flavonoid pigment, or a porphyrin pigment or the like may be used. That is, any pigment which reacts with the putrefaction component A may be used.

In this embodiment, the freshness label is formed in a structure in which a component B is not directly in contact with food. Further, when the freshness label is used for the purpose of managing freshness in a distribution stage by sampling a part of food, in general, a chemical treated as a pH indicator may be used.

For the pH indicator, for example, malachite green, thymol blue, methyl yellow, bromophenol blue, methyl orange, bromocresol green, methyl red, bromocresol purple, bromothymol blue, phenol red, naphtholphthalein, phenolphthalein, thymolphthalein or the like may be used.

The component B may be used in the form of a single kind of component or in the form of a mixture made of plural kinds of components.

(Holding Body C which Holds Component B)

The holding body C which holds the component B according to this embodiment is not particularly limited provided that the holding body C can hold the component B. However, by taking into account that the holding body C may be in contact with food to which the freshness label is applied, it is preferable that the holding body C is formed of a paper medium that is excellent in oil resistance, water resistance, antibacterial property, liquid retaining property, strength, and food peelability. The holding body C may be a medium made of the same material as a plastic wrap which covers food such as Saran Wrap® or a tray on which food is placed. It is preferable that such a medium has excellent oil resistance, water resistance, antibacterial property, liquid retaining property, strength, and food peelablity. As a matter of course, a material having no toxicity is used for the holding body C. The same goes for a printing ink for forming images on a freshness label other than the component B.

(Examples of Using Freshness Label)

The freshness label according to this embodiment is used in a state where the freshness label is placed near food of which freshness a consumer wants to know.

FIG. 2 illustrates a usage example of the freshness label according to this embodiment. As illustrated in FIG. 2, in a state where a certain food P is placed on a container T such as a tray or a dish, the freshness label 10 is placed next to the food P.

When food becomes less fresh so that the state of the food is shifted to a putrefaction state, a decomposed product (putrefaction component) of the food which is produced along with the putrefaction is generated. For example, when the food contains protein, an amine derivative, which is a cause of putrefactive odor, is generated as the decomposed product. In this embodiment, the generated decomposed product and the component B on the freshness label 10 react with each other. By recognizing a change in color or detecting a change in spectroscopic characteristics of a product formed by the chemical reaction, a user may know a freshness state of the food.

It is preferable that the freshness label 10 be placed in a hermetically sealed space where a food P preserved on a container T is covered by a plastic wrap film such as Saran Wrap® or a lid. By using the freshness label 10 in the hermetically sealed space, a sensitivity of the component B with respect to the decomposed product is increased thus enabling a user to accurately know a freshness state of the food.

With respect to the freshness label 10, colors of the printed regions No. 1, No. 2, No. 3 are changed in a stepwise manner as a freshness state of the food P becomes deteriorated. For example, as illustrated in FIG. 1(a) and FIG. 1(b), when color of the region No. 1 is changed to green from yellow, such a change indicates that freshness of the food is started to be lowered. As illustrated in (c) of FIG. 1, when color of the region No. 2 becomes green, such a change indicates that a putrefaction state is progressed so that freshness of the food is lowered and, at the same time, taste of the food is lowered. The change in color of the region No. 2 indicates that although the food is consumable at this level, it is better to eat the food as soon as possible. When color of the region No. 3 becomes green, such a change indicates that the food is no longer in a consumable state so that the food should be discarded immediately.

(Method of Preparing Freshness Label)

Hereinafter, a method of preparing the freshness label 10 illustrated in FIG. 1 is explained.

Firstly, bromothymol blue (BTB) selected as a component B is dissolved in ethanol, and three kinds of inks, of which concentration of the contained BTB is 1%, 0.5%, 0.2% respectively, are prepared. Further, KOH is added to respective three inks to adjust pH of the three inks. The pH of the ink of which concentration of the contained BTB is 1% is set to less than 2.0. The pH of the ink of which concentration of the contained BTB is 0.5% is set to less than 4.0. The pH of the ink of which concentration of the contained BTB is 0.2% is set to less than 7.0. Proper regions No. 1, 2, 3 (circular shapes in FIG. 1) are formed on a paper medium selected as the holding body C using prepared three kinds of inks. The region No. 1 is a region formed using the ink of which concentration of the contained BTB is 0.2%, the region No. 2 is a region formed using the ink of which concentration of the contained BTB is 0.5%, and the region No. 3 is an region formed using the ink of which concentration of the contained BTB is 1%. Then, ethanol is dried thus forming the freshness label 10.

The regions are formed by inkjet printing. Three kinds of regions are orange-colored regions having different BTB concentrations. When amine as the putrefaction component A is generated, a color of the region 1 tends to change first so that putrefaction may be detected at an initial level. Then, the color changes to muddy green from orange and, thereafter, changes to light blue. When a change in color of the region No. 3 is recognized, the color changes to dark blue so that it can be determined that food is in a putrefaction state at which the food is non-consumable.

(Experimentation of Putrefaction State)

The freshness label 10 prepared by the above-mentioned method was applied to actual food, and the manner how a freshness state was changed was checked.

As examples of food, meat and fish, which are commercially-available, were used. The respective foods are placed on trays configuring containers 20. The freshness label 10 is placed near the respective foods. Each tray including food is hermetically sealed by a resin film (Saran Wrap® as a whole, and the trays are preserved at a room temperature without applying any treatment.

TABLE 1

| | The number of preservation days | | | | | | | | | | | |
|---|---|---|---|---|---|---|---|---|---|---|---|---|
| | First day | | | Third day | | | Fifth day | | | Seventh day | | |
| Mark | No. 1 | No. 2 | No. 3 | No. 1 | No. 2 | No. 3 | No. 1 | No. 2 | No. 3 | No. 1 | No. 2 | No. 3 |
| Meat | Good | Good | Good | Good | Good | Good | Bad | Good | Good | Bad | Bad | Good |
| Fish | Good | Good | Good | Bad | Good | Good | Bad | Bad | Good | Bad | Bad | Bad |

Table 1 illustrates results of a change in color of the freshness label 10 with time. The evaluation criteria are set in accordance with the presence or non-presence of a change in color of each region. When a change in color of the region was recognized, the evaluation “Bad” was used, while when no change in color of the region was recognized, the evaluation “good” was used. From the results, it is understood that the degree of progress of a putrefaction state differs depending on a kind of food. With respect to fish, a change in color of the region No. 3 is confirmed on the seventh day. Accordingly, it is understood that fish is brought into a state at which the fish should not be eaten on the seventh day. With respect to meat, a change in color of the region No. 2 is confirmed on the seventh day. Accordingly, it is understood that meat maintains a state at which meat is consumable at this point of time.

When the correspondence between a consumption limit date of meat used in the above-mentioned test and color change timing of the freshness label 10 is compared with the correspondence between a consumption limit date of fish used in the above-mentioned test and color change timing of the freshness label 10, the consumption limit date of meat can be determined to be the fifth day, and the consumption limit date of fish can be determined to be the third day. Although it is merely one of criteria, with respect to fish, it can be determined that eating fish on the seventh day where color of the region No. 3 is changed is dangerous, and fish is consumable until the fifth day when color is changed to the region No. 2. In this manner, with the use of the freshness label of this embodiment, the freshness state of fish may be determined in a stepwise manner; hence, a consumer may know timing of eating fish more accurately. On the other hand, with the use of the freshness label according to this embodiment, the consumer may also know timing of discarding food more accurately; hence, the freshness label 10 may also contribute to the reduction of an amount of food to be discarded.

To take into account the taste of fish, it is desirable that fish used in the above-mentioned test is eaten before the third day which is a consumption limit date. The freshness label according to this embodiment may cope with such a demand by adjusting a color change timing of the freshness label to the consumption limit date by changing sensitivity of the component B with respect to the putrefaction component A or the like.

(Method of Checking Freshness Label)

When the freshness label according to this embodiment is used, a person can recognize a freshness state of food by checking color of the regions on the holding body C. However, it is not always necessary that the color of the regions is visually recognizable by human eyes. Instead, regions formed on the holding body C may be recognizable using a spectrometer or a camera using light having a wavelength in a non-visible light region such as ultraviolet light or infrared light or terahertz wave besides visible light.

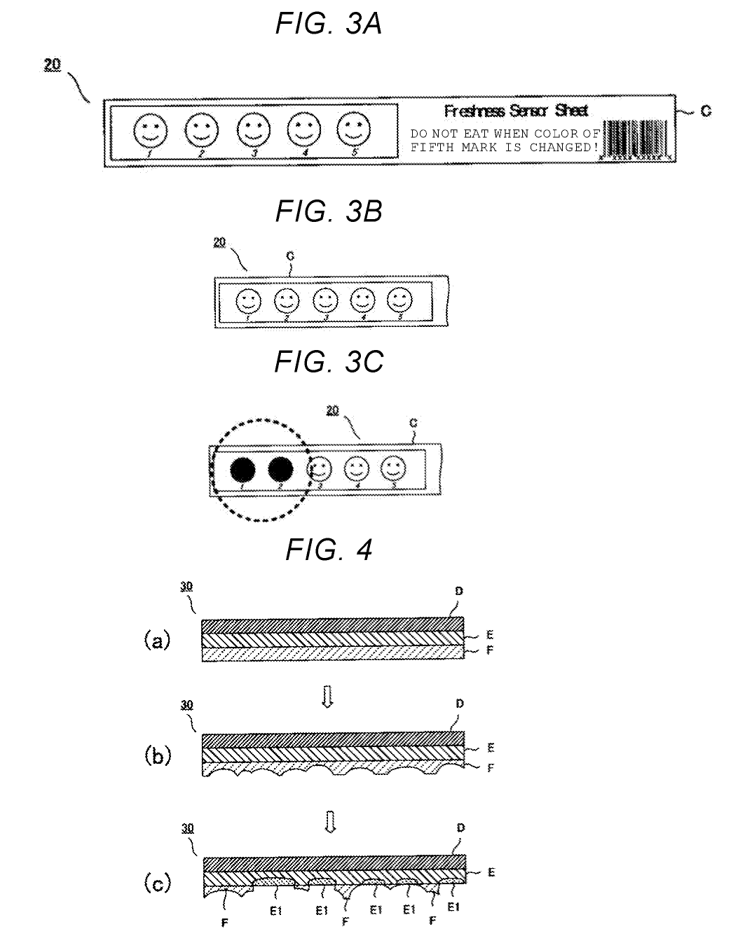

FIG. 3A to FIG. 3C illustrate one example of a freshness label where regions are recognizable using light in a non-visible light region. As illustrated in FIG. 3A to FIG. 3C, a change in color of the regions on the freshness label 20 is not visually recognizable by human eyes both at a freshness maintaining time (a) and a putrefaction progressing time (b). On the other hand, when the regions on the freshness label 20 are observed using the above-mentioned spectrometer or camera, a change in the regions may be observed as illustrated in FIG. 3C.

When the putrefaction component A and the component B react with each other, a chemical change occurs. In this method, a putrefaction state is detected by receiving a particular spectroscopy spectrum outside a visible light region which is generated when the chemical change occurs. This method is useful when a change in color in color generation or decoloring in a visible light region is small or it is desirable to increase sensitivity of the putrefaction component A and the component B. This method is also useful when the direct observation of freshness or a putrefaction state with the human eyes is not desirable. For a camera used in the method, for example, a terahertz camera can be used. As the terahertz camera, a high-speed Sub-THz imaging camera made by TOKYO INSTRUMENTS, INC., a near-infrared camera, a mid-infrared camera or a far infrared camera made by Xenics, a hyperspectral camera made by EBA JAPAN CO., LTD., or a near-ultraviolet camera made by JAI may be used.

(Other Embodiments of Freshness Label)

FIG. 4 illustrates a freshness label 30 according to another embodiment. As illustrated in FIG. 4(a), the freshness label 30 has the three-layer structure including a base material layer D, a layer E which includes the component B, and a mask layer F which masks the layer E.

It is preferable that the base material layer D is made of a material which prevents a putrefaction component A from permeating or infiltrating into the holding layer E which includes the component B. For example, it is preferable that the base material layer D is formed of a resin-made sheet made of a general-purpose material such as PET, PE, PP or PPS. Among these general-purpose materials, a material having the crystalline structure is preferably used. A metal sheet made of aluminum or the like may be also used for forming the base material layer D.

The holding layer E which includes the component B may be formed by impregnating the component B into the paper medium such as the holding body C using a printing method or the like, or may be formed by impregnating the component B into a mesh-shaped resin material or a resin material in a non-woven fabric state. The base material layer D and the holding layer E are adhered to each other with a proper adhesive agent.

The mask layer F, which masks the holding layer E, is formed of a material which reacts with a putrefaction component A and is dissolved so as to expose the holding layer E with a lapse of time or depending on a temperature state, a vapor density or the like. For example, when the putrefaction component A is an amine component, the mask layer F may be formed of a novolac resin which is an alkali-soluble resin.

The novolac resin has the alkali-soluble linear structure, and may be prepared by making phenol or a cresol mixture and formaldehyde react with each other by heating in the presence of an acid. By changing the reaction condition or the like, a novolac resin having different molecular weight or intramolecular structure may be also formed. Such a novolac resin is formed into a thin layer on a surface of the holding layer E which holds the component B, as the mask layer F. The freshness label 30 is configured such that the holding layer E is disposed between the base material layer D and the mask layer F.

As illustrated in FIG. 4(b), when the component A (not illustrated) reaches the mask layer F, the mask layer F starts to dissolve. When the generation of the component A is continued, the dissolving of the mask layer F progresses and, eventually, the dissolving of the mask layer F reaches the holding layer E which includes the component B as illustrated in FIG. 4(c). At this point of time, the putrefaction component A and the component B react with each other so that color of the holding layer E is changed or the holding layer E is decolored (E1 in the drawing, the same definition applicable hereinafter). Accordingly, the lowering of freshness of food may be visually recognized; that is, the lowering of freshness of food may be detected.

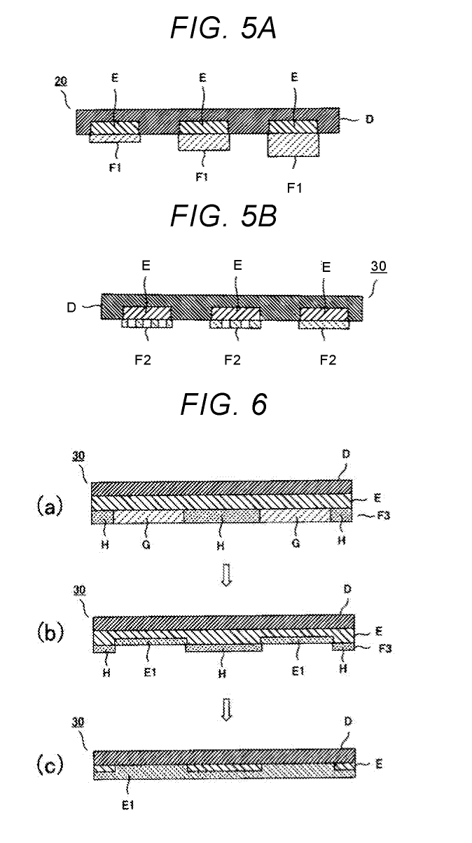

As another embodiment, as illustrated in FIG. 5A, a freshness label 20 is configured to include mask layers F1 having different thicknesses and holding layers E which include the component B in regions corresponding to the mask layers F1 so that timing at which the putrefaction component A reaches the component B may be different.

As illustrated in FIG. 5B, a freshness label 30 may be configured to include mask layers F2 having different porosities respectively so that timing at which the putrefaction component A reaches the component B may be changed. In the same manner, as illustrated in (a) of FIG. 6, a freshness label 30 may be also configured to include a mask layer F3 which has both novolac resin layers G and layers H having different molecular weights and different intramolecular structures so that timing at which the putrefaction component A reaches the component B may be also different.

The freshness labels 20, 30 have the above-mentioned configuration, and a freshness state of food may be detected in a stepwise manner. For example, As an amount of putrefaction component A generated from certain food is increased along with the progress of a putrefaction state, in the embodiment illustrated in FIG. 5A, a color of the component B changes at a portion of the mask layer F having a smaller thickness first and then the mask layer F having a larger thickness. In the embodiment illustrated in FIG. 5B, by changing a porosity of the mask layer F, a portion of the mask layer F having larger porosity starts the generation of color or decoloring prior to other portions of the mask layer F. In the embodiment illustrated in FIG. 6(a), regions having different alkaline dissolution rates such as the resin layers G and the resin layers H are disposed in the mask layer F3. Accordingly, because of the difference in the alkaline dissolution rate, as illustrated in FIG. 6(b) and FIG. 6(c), a time at which the holding layer E is exposed may be different in a stepwise manner so that the freshness label may exhibit the generation of color or the decoloring in a stepwise manner.

As a material for forming the mask layer F other than the novolac resin, for example, an alkali-soluble resin such as an acrylic resin, a PVP (polyvinyl phenol) resin, an epoxy methacrylate resin, an isobutylene-maleic anhydride copolymer resin, or a PVA (polyvinyl alcohol) resin may be used. The mask layer F may be also formed of collagen which is protein extracted from a living body, shellac, cellulose acetate phthalate which is used for pharmaceutical formulation or the like.

As another embodiment, a freshness label may indicate a freshness state of food in a stepwise manner using contrast or difference from background color.

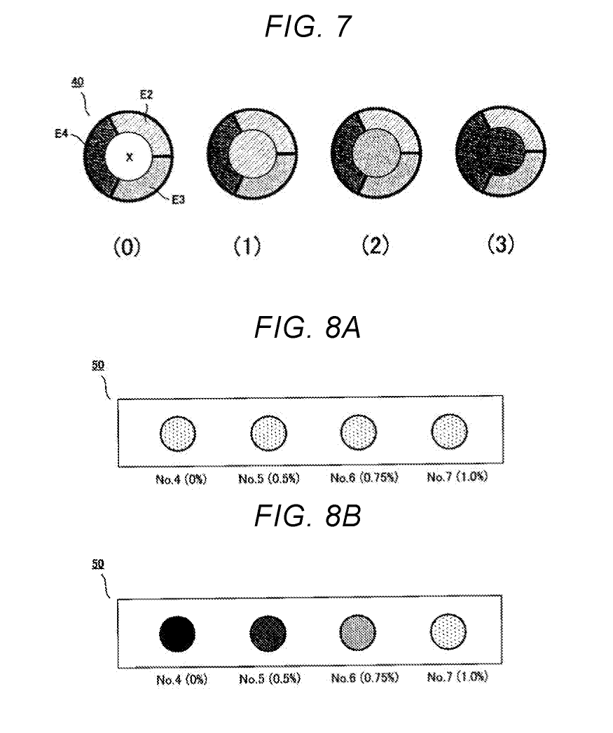

As illustrated in FIG. 7, a freshness label 40 is configured such that an ink region is formed at a center portion of a circular shape, and a plurality of regions, each having a color of the ink region at a different level in a process of reacting with the putrefaction component, are formed at a circumferential portion of the circular shape.

The ink region X at the center portion is formed using an ink where 1% of BTB is dissolved in ethanol or the like. The region at the circumferential portion includes a region E2 corresponding to a first stage, a region E3 corresponding to a second stage and a region E4 corresponding to a third stage. The regions E2 to E4 correspond to putrefaction states. Before the freshness label 30 is used or when the freshness label 30 is in an initial use state, the color of the region formed at the center portion is different from the colors of the regions E2, E3 and E4, which are formed at the circumferential portion (zero stage (0)). When the putrefaction state of food progresses, the color of the region X formed at the center portion is gradually changed and, eventually, the color of the region X agrees becomes the same as the color of the region E2 at the first stage (1) formed at the circumferential portion. Thereafter, when the putrefaction state further progresses so that a generation amount of putrefaction component is increased, the color of the region X formed at the center portion becomes the same as the color of the region E3 at the second stage (2) formed at the circumferential portion; and, eventually, the color of the region X becomes the same as the color of the region E4 at the third stage (3).

In this manner, a user may recognize a freshness state of food by comparing the color of the region X at the center portion of the freshness label 40 with colors of the portions at the circumferential portion of the freshness label 40.

As another embodiment, a change in color which indicates a putrefaction state may be made stably and in a stepwise manner by adding an acid component, a basic component, or a pH adjusting agent to an ink.

For the pH adjusting agent, sodium citrate, calcium hydrogen-phosphate, calcinated calcium, ammonium chloride, sodium hydrogen carbonate, potassium carbonate or the like which is used in a food additive or the like may be used. For the acid component, a citric acid, a glacial acetic acid, a DL-malic acid, tartaric acid or the like which is used as acidulant or the like, an L-ascorbic acid or the like which is used as another food additive may be used.

FIG. 8A and FIG. 8B illustrate the evaluation made using a freshness label to which a pH adjusting agent is added.

Firstly, solutions are prepared by adding 1% of BTB to an ethanol solvent, and 1%, 0.75% and 0.5% of an citric acid are added to the solutions respectively as a pH adjusting agent; as a result inks are prepared. Regions are formed with such inks on a paper configuring the holding body C by inkjet printing.

A freshness state indicating property was evaluated using a solution containing putrefaction component and the freshness label 50 prepared as described above. As illustrated in FIG. 8A and FIG. 8B, it was found that a color of the ink may be changed depending on the concentration of the ph adjusting agent. That is, when certain food starts to be putrefied so that a putrefaction component is gradually generated, the color of a region No. 4, to which no citric acid is added, started change first. Thereafter, when a putrefaction state progresses so that an amount of putrefaction component is increased, the color of a region No. 5, to which 0.5% of citric acid is added, started to change. Then, the color of a region No. 6, to which 0.75% of citric acid is added, started to change. Finally, the color of a region No. 7, to which 1% of citric acid is added, started to change. That is, as the putrefaction state progresses, the colors of the regions are changed in the above-mentioned order.

In this manner, by adjusting an amount of the pH adjusting agent to be added, a putrefaction state may be stably indicated in a stepwise manner.

In this manner, the freshness label according to the exemplary embodiment is a freshness detection section by which a person may know a freshness state of food in a stepwise manner. Accordingly, the person may easily know whether the food is consumable or the food should be discarded. A freshness label in the related art is a section which informs a putrefaction state using a change in color. However, different from the present disclosure, the freshness label in the related art does not indicate a quantity of freshness state in a stepwise manner. With the use of the freshness label of the exemplary embodiment, safety of food may be recognized more reliably and it is possible to contribute to the reduction of an amount of food waste.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A freshness indicating label, comprising:
a base member having a first region, a second region, and a third region, each of the first, second, and third regions containing a reactive material that changes a chemical state thereof by chemical reaction with a food-derived material that is generated as a food product putrefies,
a concentration of the reactive material contained in the first region being less than a concentration of the reactive material contained in the second region by a first value, and the concentration of the reactive material contained in the second region being less than a concentration of the reactive material contained in the third region by a second value that is different from the first value.

2. The freshness indicating label according to claim 1, wherein
the reactive material contains bromothymol blue (BTB).

3. The freshness indicating label according to claim 2, wherein
each of the first, second, and third regions further contains KOH as a pH adjusting agent.

4. The freshness indicating label according to claim 1, further comprising:
a mask layer that is formed on the first, second, and third regions of the base member and decomposable by reaction with the food-derived material.

5. The freshness indicating label according to claim 1, wherein
the first, second, and third regions are consecutively arranged along a line in this order.

6. The freshness indicating label according to claim 1, wherein
a color of the reactive material changes when the reactive material reacts with the food-derived material.

7. The freshness indicating label according to claim 6, wherein
the base member further has a fourth region in which information indicating what the changed color of the reactive material signifies.

8. The freshness indicating label according to claim 7, wherein
the base member further has a fifth region in which a product identification code is printed.

9. A method for indicating freshness of a food product comprising:
forming, on a base member, a first region, a second region, and a third region, each of the first, second, and third regions containing a reactive material that changes a chemical state thereof by chemical reaction with a food-derived material that is generated as the food product putrefies, a concentration of the reactive material contained in the first region being less than a concentration of the reactive material contained in the second region by a first value, and the concentration of the reactive material contained in the second region being less than a concentration of the reactive material contained in the third region by a second value that is different from the first value; and
enclosing the food product with the base member having the first, second, and the third regions in a closed space.

10. The method according to claim 9, wherein
the reactive material contains bromothymol blue (BTB).

11. The method according to claim 10, wherein
each of the first, second, and third regions further contains KOH as a pH adjusting agent.

12. The method according to claim 9, further comprising:
forming, on the first, second, and third regions of the base member, a mask layer that is decomposable by reaction with the food-derived material.

13. The method according to claim 9, wherein
the first, second, and third regions are consecutively arranged along a line in this order.

14. The method according to claim 9, wherein
a color of the reactive material changes when the reactive material reacts with the food-derived material.

15. The method according to claim 14, further comprising:
forming information indicating what the changed color of the reactive material signifies on a fourth region of the base member.

16. The method according to claim 15, further comprising:
forming a product identification code on a fifth region of the base member.
